(19) United States
(12) Patent Application Publication
(10) Pub. No.: US 2008/0153907 A1
(43) Pub. Date: Jun. 26, 2008

(54) Pharmaceutical Composition for the Treatment of Cancer Comprising Lhm-Ra Complex

(75) Inventors: Jin-ho Choy, Seoul (KR); Taeun Park, Seoul (KR); Sang-tae Kim, Seoul (KR); You-hwan Son, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: NANOHYBRID CO., LTD., Seoul (KR)

(21) Appl. No.: 11/817,101

(22) PCT Filed: Feb. 22, 2006
(86) PCT No.: PCT/KR2006/000600

§ 371 Date: Aug. 24, 2007

(30) Foreign Application Priority Data
Feb. 25, 2005 (KR) 10-2005-0016168

(51) Int. Cl. A61K 31/19; A61P 35/00

(52) U.S. Cl. 514/557

(57) ABSTRACT

Provided is a pharmaceutical composition for the treatment of liver cancer, including a layered metal hydroxide-retinoic acid (LMH-RA) hybrid as a novel drug delivery system which shows few side effects of retinoic acid, good drug stability, sustained drug release, and improved drug delivery efficiency.

### CROSS-REFERENCE TO RELATED PATENT APPLICATION(S)

This application is a 35 U.S.C. § 371 National Phase Entry Application from PCT/KR2006/000600, filed Feb. 22, 2006, and designating the United States. This application also claims the benefit of Korean Patent Application No. 10-2005-0016168, filed on Feb. 25, 2005, in the Korean Intellectual Property Office, the disclosure of which is incorporated herein in its entirety by reference.

### BACKGROUND OF THE INVENTION

1. Field of the Invention

The present invention relates to a layered metal hydroxide-retinoic acid (LMH-RA) hybrid and its anticancer efficacy. More particularly, the present invention relates to a pharmaceutical composition for the treatment of cancers, including a hybrid of RA and LMH which is an inorganic carrier.

2. Description of the Related Art

Generally, layered inorganic compounds can include various materials in their interlayers. For example, various functional guest materials can be intercalated into the interlayers of aluminosilicates, metal phosphates, etc., using layer charges generated by isomorphous substitution of metal ions constituting host lattice layers or physicochemical adsorption capability induced by layer surface modification. In addition, it is known that a pore size of crosslinked clay, MCM-41, etc. are adjusted to physically adsorb molecules of a predetermined size. Among these layered inorganic compounds, layered double hydroxides (LDHs), also called “anionic clays”, are composed of positively charged metal hydroxide layers, interlayer anions capable of compensating for the positive charges, and interlayer water. It is known that various anions can be easily introduced into the interlayers of LDHs using ion-exchange reaction or coprecipitation. These LDHs and their derivatives have received much interest due to the technical importance of layered nano-hybrids in catalytic reactions, separation technology, optical industry, medical engineering, pharmaceutical industry, etc., and thus, research thereon has been actively conducted.

For example, the structures of interlayer anions (carbonate) and water in hydrotalcite ([Mg₃Al(OH)₈]+[0.5CO₃.mH₂O]⁻— a mineral name of a compound having a magnesium (Mg)-aluminum (Al)-based LDH structure—were elucidated using ¹H and ¹³C NMR spectra [“Ordering of intercalated water and carbonate anions in hydrotalcite—An NMR study”, A. van der Vol. et al., Journal Physical Chemistry, 1994, 98, 4050-4054].

Sang-Kyeong Yun et al. [“Layered double hydroxides intercalated by polyoxometalate anions with Keggin(α-H₂W₁₂O₄₀⁶⁻), Dawson(α-P₂W₁₈O₆₂⁶⁻), and Finke(CO₄(H₂O)₂(PW₉O₃₄)₂¹⁰⁻) structures”, Inorganic Chemistry, 1996, 35, 6853-6860] disclosed the pillaring of Mg₃AI LDH by polyoxometalate (P₂W₁₈O₆₂⁶⁻ or CO₄(H₂O)₂(PW₉O₃₄)₂¹⁰⁻) using ion exchange reaction of LDH-hydroxide and -adipate precursors with the polyoxometalate, and evaluation results of structural and thermal properties of the resultant LDH. Ji-Won Moon et al. [“Crystal structures of some double hydroxide minerals”, Mineralogical Magazine, 1973, 39[304], 377-389] disclosed the structural characteristics of some LDHs, and the types and structures of metal cations and interlayer anions available for the LDHs.

F. Cavani et al. [“Hydrotalcite-type anionic clays: Preparation, properties and applications”, F. Cavani et al., Catalysis Today, 1991, 11, 173-301] comprehensively reviewed the historical background, available components (e.g., types of metal cations and interlayer anions), structural properties, and applications of LDHs. In contrast, the incorporation of biological materials into LDH is not much known except for those phosphate ion-containing biological materials, such as DNAs or RNAs (Korean Patent No. 10-0359716).

Recently, retinoid derivatives (e.g., retinols, retinoic acids, etc.) have received much interest as materials of functional cosmetic products for skin whitening, the removal or prevention of pigmented lesions such as melasma and freckles, and anti-wrinkle effect due to intrinsic antioxidative activity. However, these retinoid derivatives are very unstable to be destroyed in the air, which causes great restriction in handling of them and their applicability. In particular, retinoids such as vitamin A (retinol), known as anticancer materials, cause serious side effects, such as skin irritation, when administered in high dosage for anticancer therapy, and thus, are practically inapplicable. U.S. Pat. No. 4,310,546 discloses an N-(4-acyloxyphenyl)-all-trans-retinamide compound, U.S. Pat. No. 4,323,581 discloses N-(4-hydroxyphenyl)-all-trans-retinamide, and U.S. Pat. No. 4,665,098 discloses N-(4-hydroxyphenyl)retinamide (known as fenretimide).

It is known that retinoids are involved in cell differentiation and development by inducing dimerization of nuclear receptors, RAR (retinoic acid receptor) and RXR (retinoid X receptor) to promote the entry of RAR/RXR into cell nuclei [Dino moras et al., Nature, 1995, 375, 377-382]. It is also known that retinoids exhibit anticancer effects by indirectly regulating the activity of a transcriptional activation factor participating in tumorigenesis and metastasis, i.e., AP-1 (activation protein-1), so that the expression of a target gene of AP-1 is suppressed [Yang-Yen H. F. et al., New Biol. 3: 1206-1219, 1991]. It is also known that retinoids including retinol can inhibit uncontrolled cell proliferation and induce differentiation or apoptosis, and thus, can be effectively used for the treatment or prevention of cancers [Hong W. K. and Itri L. M., Biol. Chem. Med., 2nd ed. edited by Sporn et al., New York: Raven Press; 597-630, 1994]. However, the use of retinoids may produce side effects, such as skin irritation, toxicity in organ systems, and deformation, by some proteins which are activated by the interaction between the retinoids and their receptors [Hathcock J. N. et al., Am. J. Clin. Nutr., 52, 183-202, 1990]. Recently, some retinoid derivatives with better anticancer effects and fewer side effects than existing retinoids have been reported. However, when these retinoid derivatives are administered in the form of retinoid-based drugs in high dosage for anticancer therapy, irritation to tissues may be caused. Thus, it is necessary to reduce a dosage of the retinoid derivatives, which limits the use of the retinoid derivatives as anticancer drugs. Retinoids exhibit low tissue distribution due to low solubility, and thus, the use of high-dose retinoids is needed. In view of this problem, LDH-retinoic acid (RA) was suggested.

Currently available drugs for the treatment of liver cancer include injectable forms of 5-fluorouracil (5-FU), cytarabine, and alkyloxane, which are described in the Korean pharmacopoeia. However, these drugs contribute to prevent the proliferation of cancer cells, rather than to induce the death of cancer cells, and thus, are not effective for the fundamental treatment of liver cancer. With respect to a holmium-166-chitosan complex (DW-166HC), known as a potent treatment of liver cancer, its clinical safety and effects have not been completely evaluated, and thus, long-term clinical trials with many patients must be performed. Furthermore, in a case where two or more tumor masses are distributed over several organs, tumors spread to distant organs (metastasis), patients suffer from abdominal dropsy or jaundice, or several blood vessels extend into a tumor mass, chemotherapy with DW-166HC cannot be used. In addition, the chemotherapy with DW-166HC must be prescribed and managed by a medical doctor.

There are a few foreign and domestic patents which are more or less associated with LDH-based nanocomposites, in particular, LDH-RA. LDH may be a natural or synthetic LDH. A method of synthesizing LDH is disclosed in U.S. Pat. Nos. 3,539,306 and 3,650,704. In particular, Korean Patent Application No. 10-2002-0047318 discloses a hydrozincite-3-benzoyl-α-methylbenzene acetic acid hybrid, Korean Patent Application No. 10-2001-0046774 discloses a vitamin-LDH hybrid wherein anionic vitamins or their derivatives are intercalated into interlayers of LDHs which works as inorganic carriers, and the method of preparing the same, and Korean Patent Application No. 10-1993-0002369 discloses a UV-screening composition suitable for human skin. However, these patent documents are silent about the anticancer efficacy of LDH-RA.

It is very difficult to develop a treatment for liver cancer considering the fact that the liver participates in all metabolisms of the human body. Thus, a LDH-RA hybrid, developed by the present inventors, which is a selective anticancer active material capable of exhibiting minimal toxicity in normal cells and maximal anticancer activity in liver cancer cells, can be used as a potent treatment of liver cancer.

### SUMMARY OF THE INVENTION

In view of the above problems, the present invention provides a pharmaceutical composition for the treatment of liver cancer, including a retinoic acid-layered metal hydroxide (RA-LMH) hybrid as a novel drug delivery system which shows few side effects of RAs, good drug stability, sustained drug release, and improved drug delivery efficiency.

The present invention is directed to prepare a retinoic acid-layered metal hydroxide (RA-LMH) hybrid wherein RA is intercalated into the interlayer of LMH by anion exchange reaction. RA is very unstable and toxic, and thus, involves problems such as antigenic effects in immune response. Thus, a novel drug delivery system for RA has been required. LMH is soluble in an acidic condition but very stable in a neutral or basic condition. In this regard, LMH is expected to be a novel drug delivery system capable of conferring stability and sustained release property to RA. Metal hydroxide used in the RA-LMH hybrid according to the present invention is harmless to human body, and the release of RA from LMH can be appropriately adjusted. The RA-LMH hybrid according to the present invention has a significant meaning since it is a first attempt to apply to a pharmaceutical composition for cancer treatment. Therefore, it is an objective of the present invention to provide a RA-LMH hybrid which stabilizes unstable retinoid derivatives, extends effect of RA through sustained-release of it, and induces the apoptotic cell death of tumor cells.

According to an aspect of the present invention, there is provided a pharmaceutical composition for the treatment of a cancer, including an LMH-RA hybrid as an effective ingredient. The pharmaceutical composition can be used for the treatment of various cancers due to the anticancer activity of RA [Yang-Yen H. F. et al., New Biol. 3: 1206-1219, 1991, Hong W. K. and Itri L. M., Biol. Chem. Med., 2nd ed. edited by Sporn et al., New York: Raven Press; 597-630, 1994]. However, the following working examples of the present invention have demonstrated that the pharmaceutical composition of the present invention is particularly useful for the treatment and prevention of liver cancer.

The LMH may be layered double hydroxide (LDH) or hydroxy double salt (HDS). Although the LDH and HDS are similarly prepared by titrating a metal salt-containing solution with a base solution, the HDS contains a single metal element such as a divalent metal element, whereas the LDH contains two or more metal elements of different valencies, usually divalent and trivalent metal elements. Thus, the LMH-RA hybrid of the present invention may be a LDH-RA hybrid or a HDS-RA hybrid.

The LDH-RA hybrid or the HDS-RA hybrid may be prepared by intercalating RA into the interlayer of LDH or HDS using ion exchange, coprecipitation, or adsorption. According to the coprecipitation method, RA is added as a reactant during synthesis of LDH or HDS, and the intercalation of RA into the interlayer of LDH or HDS occurs simultaneously with synthesis of LDH or HDS. According to the ion exchange method, anion species in the interlayer of previously synthesized LDH or HDS are substituted by RA. According to the adsorption method, anions in the interlayer of LDH or HDS are removed by thermal treatment, and RA is then intercalated into the interlayer of LDH or HDS.

The LMH-RA hybrid may be represented by Formula 1 below:

[M²⁺₁₋ₓN³⁺ₓ(OH)₂][RAⁿ⁻]_{x/n}.yH₂O [Formula 1]

wherein M²⁺ is a divalent metal cation selected from the group consisting of Mg²⁺, Ni²⁺, Cu²⁺, and Zn²⁺, N³⁺ is a trivalent metal cation selected from the group consisting of Al³⁺, Fe³⁺, V³⁺, Ti³⁺, and Ga³⁺, x is a value ranging from 0.1 to 0.5, RA is a retinoic acid or its derivative, n is a charge number of RA, and y is a positive number.

The LMH-RA hybrid may also be represented by Formula 2 below:

[M²⁺(OH)₈][RAⁿ⁻]_{2/n}.yH₂O [Formula 2]

wherein M²⁺ is a divalent metal cation selected from the group consisting of Mg²⁺, Ni²⁺, Cu²⁺, and Zn²⁺, RA is a retinoic acid or its derivative, n is a charge number of RA, and y is a positive number.

In Formula 1, the x value is related to a metal composition ratio and may range from 0.1 to 0.5, and more preferably, from 0.25 to 0.33. If the x value is outside the range, the encapsulation of RA into an inorganic LDH carrier, i.e., the intercalation of RA between the hydroxide layers of the LDH carrier may not occur, which renders the production of a desired LDH-RA hybrid difficult.

The LMH-RA hybrid of the present invention may be used in a hydrate form. The degree of hydration can be expressed as the y value. The y value can be changed according to various factors, such as moisture content in air. Generally, the y value can be represented by a positive number.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating a retinoic acid-layered double hydroxide (RA-LDH) hybrid (a) and a retinoic acid-hydroxy double salt (RA-HDS) hybrid (b);

FIG. 2 is X-ray diffraction patterns of a NO3-LDH hybrid (a), a RA-LDH hybrid (b), and a RA-HDS hybrid (c);

FIG. 3 is ultraviolet-visible (UV-Vis) spectra of a RA and a RA-LDH hybrid, and dissolution data of RA with time (UV-Vis absorbance with time when 5 mg of a RA-LDH hybrid is dispersed in an aqueous solution);

FIG. 4 shows a morphological change of hepatocarcinoma cell line, CHX, by a RA-LDH hybrid;

FIG. 5 shows the expression of fluorescein isothiocyanate (FITC) with time in the CHX hepatocarcinoma cell line;

FIG. 6 shows endocytosis of an LDH-FITC hybrid in the CHX hepatocarcinoma cell line;

FIG. 7 shows a distribution of an LDH-FITC hybrid in the Golgi region of the CHX hepatocarcinoma cell line;

FIG. 8 shows a distribution of an LDH-FITC hybrid in the lysosomes of the CHX hepatocarcinoma cell line;

FIG. 9 is a graph illustrating the activity of lactic acid dehydrogenase in the CHX hepatocarcinoma cell line;

FIG. 10 shows an effect of a RA-LDH hybrid on DNA fragmentation;

FIG. 11 is Western blotting analysis results showing an effect of a RA-LDH hybrid on protein expression;

FIG. 12 shows an effect of a RA-LDH hybrid on tumor development in xenografted nude mice; and

FIG. 13 is haematoxylin-and-eosin (H/E) staining results showing an effect of a RA-LDH hybrid on tumor development in xenografted nude mice.

### DETAILED DESCRIPTION OF THE INVENTION

The present invention will now be described more fully with reference to the accompanying drawings, in which exemplary embodiments of the invention are shown.

The present invention provides an inorganic layered metal hydroxide-retinoic acid (LMH-RA) hybrid wherein a retinoic acid or its derivative is intercalated into the interlayer of layered double hydroxide (LDH) or hydroxy double salt (HDS) used as an inorganic carrier, its anticancer effect, and a pharmaceutical composition using the LMH-RA hybrid. The LMH-RA hybrid of the present invention exhibits a pharmaceutical efficacy for tumor treatment by inducing apoptotic cell death of tumor cells.

The LMH-RA hybrid according to the present invention includes RA intercalated into the interlayer of a layered inorganic compound, such as LDH or HDS (see Examples 1 and 2). Various functional guest materials can be intercalated into the interlayer of the layered inorganic compound using layer charges generated by isomorphous substitution of metal ions constituting host lattice layers or physicochemical adsorption capability induced by layer surface modification. LDH, also called “anionic clay”, is composed of positively charged metal hydroxide layers, interlayer anions capable of compensating for the cations, and interlayer water. A LDH-RA hybrid may be represented by [M2+1−xN3+x(OH)2][An−]x/n yH2O where M2+ is a divalent cation, N3+ is a trivalent cation, and An− is an n-valent anion. The layer charge density of the LDH-RA hybrid can be adjusted by changing the ratio of the divalent cation to the trivalent cation. The n-valent anion can be easily intercalated into the interlayer of LDH using ion exchange or coprecipitation. LDH and its derivatives have received much interest due to the technical importance of layered nano-hybrids in catalytic reactions, separation technology, optical industry, medical industry, engineering, etc.

As used herein, the term “LMH-RA hybrid” is not a simple mixture but is a hybrid complex synthesized by chemical or physical interaction between components. For example, cationic LMH and an anionic active ingredient for a cosmetic product can be chemically bound by electrostatic interaction. Ion exchange and coprecipitation are methods based on chemical interaction. According to the ion exchange method, ions such as nitrate (NO3−), chlorine (Cl—), or carbonate (CO32−) in the interlayer of LMH are substituted by ionized drug molecules. According to the coprecipitation method, ionized drug molecules are added to a metal-containing solution during titration, and the encapsulation of the drug molecules occurs simultaneously with formation of LMH. Meanwhile, an adsorption method is based on physical interaction, i.e., van der Waals force between an organic material (e.g., tocopherol succinate) previously incorporated in LMH and an active component (e.g., retinol). The above-illustrated preparation examples are only for illustrative purpose, and thus not intended to limit the scope of the present invention. In practical, both electrostatic interaction and van der Waals force may exist in the LMH-RA hybrid according to components or preparation conditions.

The LMH-RA hybrid of the present invention can be formulated into pharmaceutically acceptable dosage forms in combination with a pharmaceutically acceptable additive, such as an excipient, an adjuvant, a diluent, an isotonic solution, a preservative, a lubricant, and a solubilizing aid.

A pharmaceutical composition of the present invention can be administered in the form of an adult dosage of 1 μg/kg/day to 400 mg/kg/day of the LMH-RA hybrid used as an active ingredient. An adequate dosage is determined according to the degree of disease severity.

The pharmaceutical composition of the present invention can be administered in the form of tablets, foam tablets, capsules, granules, powders, sustained-release tablets, sustained-release capsules (single unit formulations or multiple unit formulations), intravenous or intramuscular injectable ampules, suspensions, or suppositories, or in other suitable dosage forms.

In order to prepare pharmaceutical formulations using the pharmaceutical composition, the LMH-RA hybrid can be used in a pharmaceutically effective amount, in combination with a physiologically tolerated excipient and/or diluent and/or adjuvant, according to an appropriate preparation method.

Hereinafter, the present invention will be described more specifically with reference to the following working examples. The following working examples are for illustrative purposes and are not intended to limit the scope of the present invention.

### EXAMPLE 1

RA-inorganic hybrids were synthesized by coprecipitation as follows.

(1) A solution of a RA derivative in 0.2 M NaOH was dropwise added to a mixture of metal cations Zn(II) and AI(III) (1<Zn/Al<4). The resultant precipitate was centrifuged and washed to give a RA-inorganic hybrid. The entire processes were performed in a nitrogen atmosphere to prevent contaminations with CO₂ in air. The resultant compound was represented by the following formula:

M^{II}₁₋ₓAl^{III}ₓ(OH)₂(C₂₀H₂₇O₂)ₓ.mH₂O

M^{II}: Mg, Zn, Ni, . . . 0.1<x<0.5)

(2) A solution of a RA derivative in 0.2 M NaOH was dropwise added to a metal cation Zn(II)-containing solution. The resultant precipitate was centrifuged and washed to give a RA-inorganic hybrid compound. The entire processes were performed in a nitrogen atmosphere to prevent contaminations with CO₂ in air. The resultant compound was represented by the following formula:

M^{II}₅(OH)₈(C₂₀H₂₇O₂)₂.mH₂O

(M^{II}: Zn, Ni, . . . )

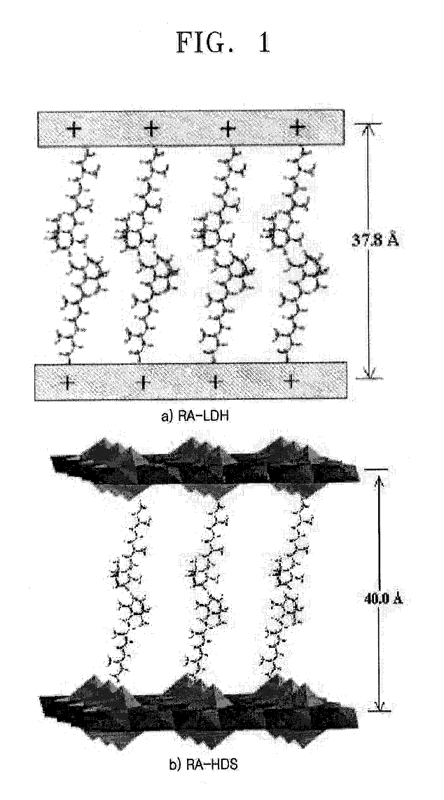

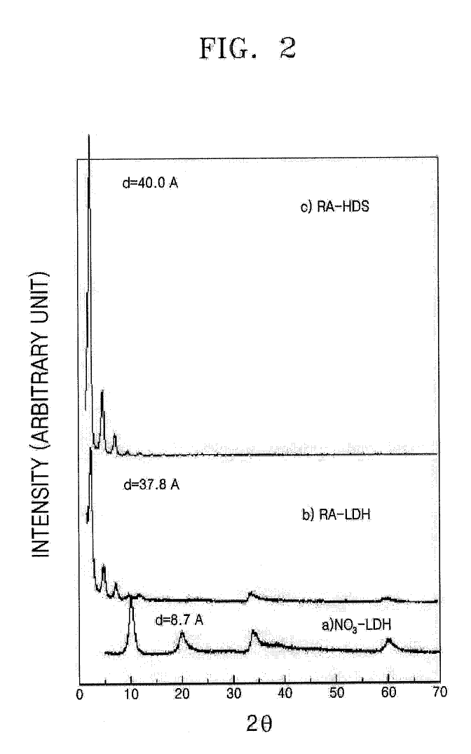

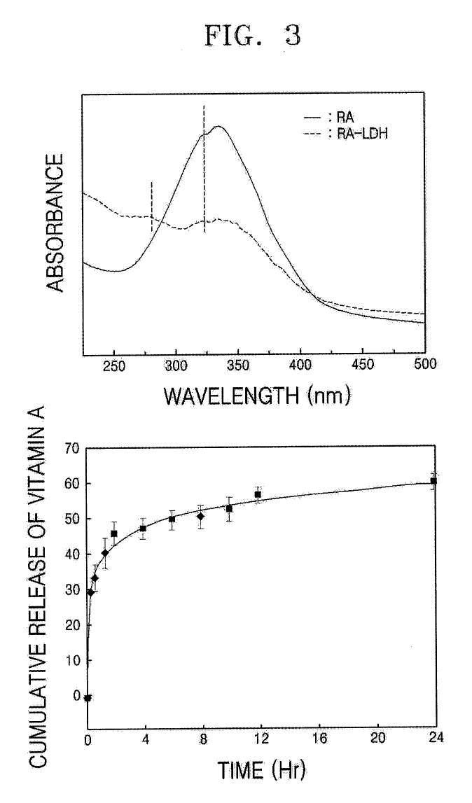

The X-ray diffraction patterns of the RA-inorganic hybrids are shown in FIG. 2 and the UV-Vis spectra of the RA-inorganic hybrids are shown in FIG. 3. Referring to FIGS. 2 and 3, the interlayer distance of the RA-inorganic hybrids corresponds to 2-fold of the molecular length of RA, and the UV-Vis spectral absorption peaks of the RA-inorganic hybrids are identical to those of RA. These results show that RAs are stabilized and vertically arranged in the interlayer of metal hydroxide layers. Based on these results, the probable arrangement of RAs between inorganic lattice layers is as shown in FIG. 1.

### EXAMPLE 2

A dispersion solution of 5 mg of a LDH-RA hybrid in 40 mL of distilled water was added to seven test tubes, incubated at 35° C. in a thermostat system rotating at 270 rpm, and centrifuged at predetermined time intervals. The UV-Vis spectra of the resultant supernatants were measured, and the results are shown in FIG. 3. Absorbance with time at the maximum absorption wavelength (288 nm) is also shown in FIG. 3. Referring to FIG. 3, 60% RA was released for 2 hours after the reaction was initiated. After then, a small amount of RA was released continuously. These results show that RA stabilized between LDH lattice layers is delivered continuously and acts on a target site.

### EXAMPLE 3

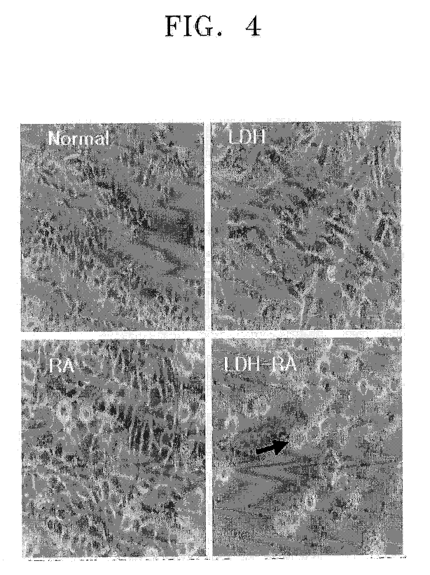

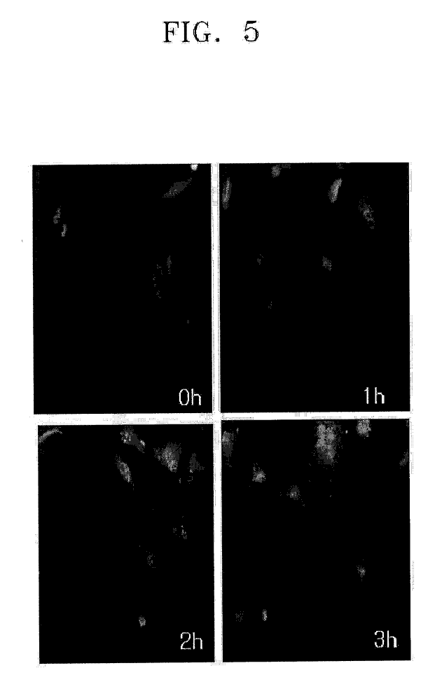

In order to examine the morphological change of tumor cell line, CHX, by LDH-RA treatment, about 10⁴ cells were seeded in each of four wells of a 6-well plate and incubated in a 5% CO₂ incubator at 37° C. One of the four wells was used as a control group with no drug treatment. The remaining three wells were treated with 40 μg/ml of LDH, 250 μg/ml of RA, and 1,000 μg/ml of LDH-RA, respectively. At 12 hours after the treatment, the morphological change of the cells in each well was observed, and the results are shown in FIG. 4. Referring to FIG. 4, in the control group, significant augmentation of cell proliferation was observed. In the LDH-dose group and the RA-dose group, cell proliferation was slightly retarded but no apoptotic cell death was observed. In the LDH-RA dose group, cell proliferation was greatly suppressed and apoptotic cell death was greatly increased. Meanwhile, in order to determine the programmed time of apoptotic cell death by LDH-RA treatment, Tunel assay was performed, and the results are shown in FIG. 5. Referring to FIG. 5, the strongest fluorescence was observed at 2-3 hours after the treatment. This result shows that LDH-RA-mediated cell death occurs at 2-3 hours after the LDH-RA treatment.

### EXAMPLE 4

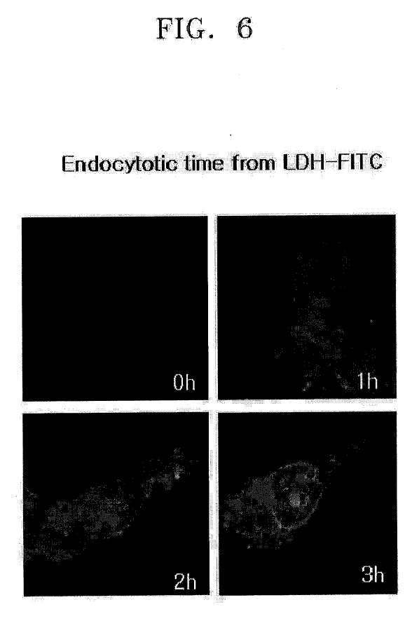

In order to evaluate an effect of a LDH-RA hybrid synthesized according to the present invention on cells, the endocytosis of LDH with time in the CHX tumor cell line was observed. For this, the CHX tumor cells were plated on cover glasses and cultured. Then, the cells were treated with previously prepared LDH-FITC (Fluorescein Isothiocyanate) so that endocytosis occurred. At this time, the cells were washed with a phosphate buffer saline (PBS) at 0, 1, 2, and 3 hours after the LDH-FITC treatment, and fixed with methanol for 10 minutes. The cover glasses were placed on slide glasses, and cellular change was observed in a dark room using a laser-scanning confocal microscope (Bio-Rad). The results are shown in FIG. 6. Referring to FIG. 6, at an initial stage (0 hours), no green fluorescence was observed in the tumor cells as well as their surroundings. However, green fluorescence started to appear at 1-2 hours after the LDH-FITC treatment, and the strongest green fluorescence was observed at 3 hours after the LDH-FITC treatment. In particular, strong green fluorescence was observed in nuclear membranes and the surroundings of endoplasmic reticula. This can be explained by the release of FITC from LDH in acidic small organelles (<pH 6) around nuclear membranes, such as endoplasmic reticula, Golgi, and lysosomes. Thus, it is thought that FITC easily reaches small organelles through LDH and is then released from LDH due to the acidic environment of the organelles.

### EXAMPLE 5

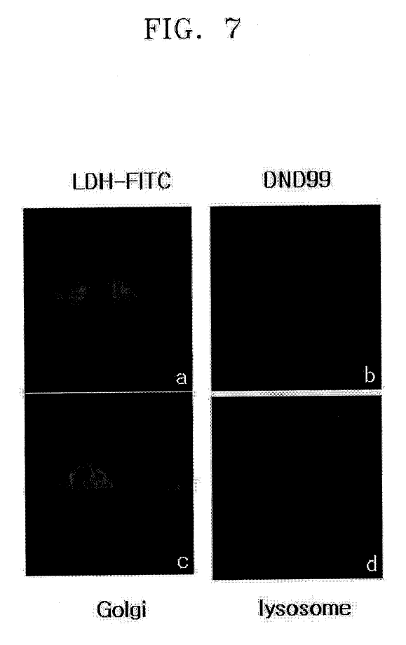

In order to determine which organelle participates in release of FITC from LDH, the distribution of FITC in the organelles of cells was observed, and the results are shown in FIG. 7. Referring to FIG. 7, LDH-FITC first reached Golgi and lysosomes around nuclear membranes after endocytosis. Thus, it is thought that FITC is released from LDH in acidic (pH<6) Golgi and lysosomes, and distributed in the small organelles and nuclear membranes of cells.

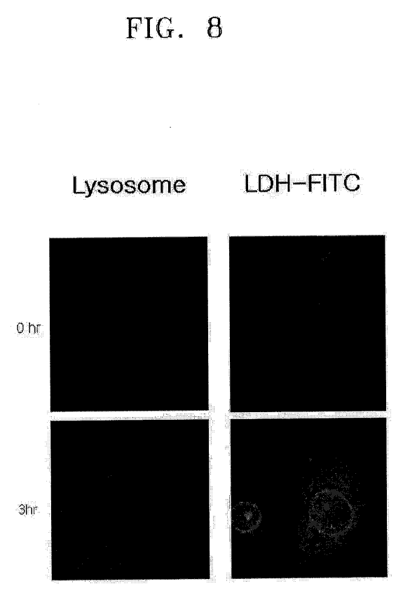

In order to determine if the release of FITC from LDH occurs in Golgi, the Golgi was stained with Alexa Fluor anti-golgi-97 antibody, and lateral fluorescence distribution was observed. As a result, green fluorescence was observed in the Golgi and the surroundings. This result shows that LDH-FITC is first ingested into the cell membrane by endocytosis and then reaches the nuclear membrane and the surrounding organelle, Golgi. This can be explained by the release of FITC from LDH due to the acidic environment of the Gogi. On the other hand, the release of FITC from LDH in lysosomes was also evaluated using lysoTracer Red DND-99. As a result, red fluorescence was observed in the lysosomes. Like in the Golgi, it is thought that after endocytotic uptake of LDH-FITC into the cells, FITC is released from LDH in lysosomes due to the acidic environment (pH<6) of the lysosomes (see FIGS. 7 and 8).

### EXAMPLE 6

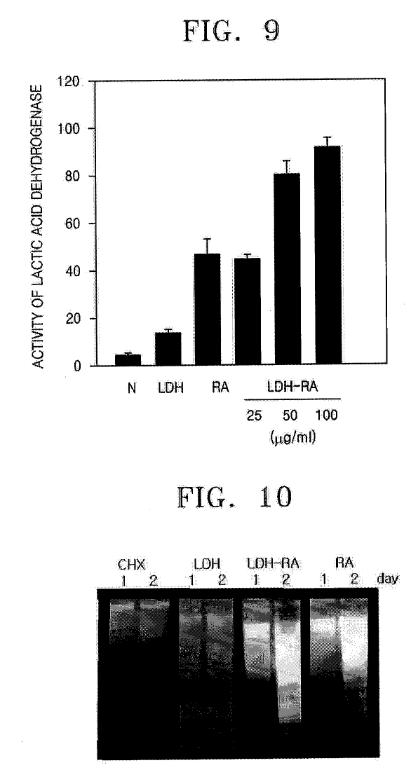

In order to evaluate an anticancer effect of a LDH-RA hybrid obtained according the present invention, activity of lactic acid dehydrogenase associated with apoptotic cell death was measured. For this, the CHX tumor cells were seeded into each well of a 96-well plate. The CHX tumor cells were divided into 6 groups: normal group with no treatment, LDH-dose group with 1,000 μg/ml of LDH, RA-dose group with 250 μg/ml of RA, LDH-RA low-dose group with 25 μg/ml of LDH-RA, LDH-RA mid-dose group with 50 μg/ml of LDH-RA, and LDH-RA high-dose group with 100 μg/ml of LDH-RA. All groups were cultured for 12 hours. 20 μl of pyruvate substrate (NADH 1 mg/ml) was added to each group, and the cultures were mixed at room temperature for 2 minutes and stirred at 37° C. for 30 minutes. 20 μl of a color reagent (Sigma 505-2) was added to each culture, and the resultant cultures were mixed at room temperature for 20 minutes. 100 μg of 0.4N NaOH was added to each culture, and the resultant cultures were mixed at room temperature for 15 minutes. Absorbance (A570/A630) of each culture was measured using an ELISA reader, and the results are shown in FIG. 9. Referring to FIG. 9, the activities of lactic acid dehydrogenase of the normal group, the LDH-dose group, and the RA-dose group were 6±1.5%, 13±2%, and 42±5%, respectively. The activities of lactic acid dehydrogenase of the LDH-RA low-dose group, the LDH-RA mid-dose group, and the LDH-RA high-dose group were 41±2%, 76±6%, and 86±5%, respectively. In particular, the activity of lactic acid dehydrogenase of the LDH-RA dose groups was 2-fold or more higher than that of the RA-dose group in the same concentration. This might be because LDH facilitates the introduction of RA into cells, and thus, a RA-mediated apoptotic pathway is increasingly activated, thereby inducing a higher apoptotic cell death than the RA-dose group.

### EXAMPLE 7

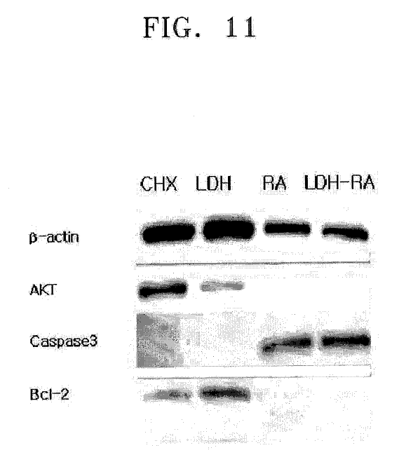

In order to determine whether a LDH-RA hybrid induces DNA fragmentation, the CHX tumor cells were seeded at 1×10⁴ cells/well in a 6-well plate and cultured for 12 hours. A LDH-dose group, a RA-dose group, and a LDH-RA dose group were treated with 1,000 μg/ml of LDH, 250 μg/ml of RA, and 40 μg/ml of LDH-RA, respectively, for 1-2 days, and cells were then collected. The cells were treated with 200 μg of a lysis buffer (10 mM Tris-HCl, pH 7.5, 1 mM EDTA, 0.2% Triton X-100) and incubated on ice for 30 minutes. Then, proteinase K (100 μg/ml) was added to the cells, followed by incubation in a 50° C. water bath for 5 hours. The resultant cultures were thoroughly mixed with a 1:1 phenol/chloroform mixture and centrifuged at 15,000 rpm for 15 minutes. The supernatants were collected and treated with 100% EtOH. The precipitates were dried, and 35 μg of RNase (50 μg/ml)-containing dH₂O was added thereto. The resultant solutions were analyzed by 1.5% agarose gel electrophoresis to qualitatively determine DNA fragmentation, and the results are shown in FIG. 10. Referring to FIG. 10, in the normal group, no apoptotic cell death was observed due to active cell proliferation (see FIG. 4 showing the morphological change of the normal cell group). In the LDH-dose group, no or few DNA fragmentation was observed. On the other hand, the RA-dose group and the LDH-RA dose group formed discontinuous ladder patterns (200-400 bp in length) by cleavage of genomic DNA into DNA fragments by endonuclease activated during apoptosis. Here, based on the observation of a 1 kb or less DNA ladder pattern, it is thought that apoptosis is induced by RA released from LDH.

### EXAMPLE 8

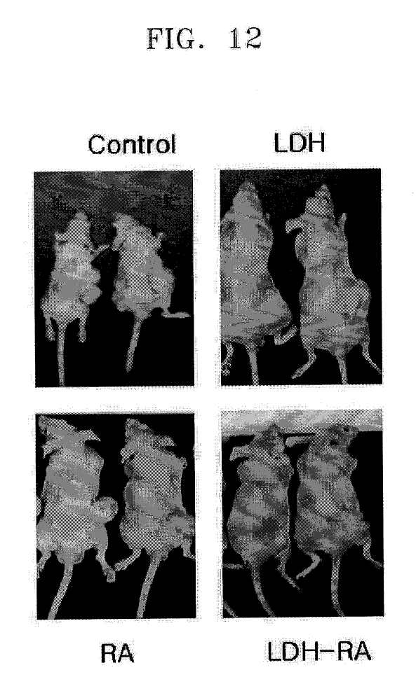

The CHX tumor cells were seeded at 1×10⁴ cells/well into four wells of a 6-well plate, and cultured for 12 hours. The four wells were used for a normal group, an LDH-dose group, a RA-dose group, and a LDH-RA dose group, respectively. The normal group was an untreatment group. The LDH-dose group, the RA-dose group, and the LDH-RA dose group were treated with 1,000 μg/ml of LDH, 250 μg/ml of RA, and 40 μg/ml of LDH-RA, respectively, for 12 hours, and cells were then collected. Then, the cells were treated with a lysis buffer (50 mM Tris-HCl pH 7.5, 1% (v/v) Triton X-100, 150 mM NaCl, 10% (v/v) glycerol, 2 mM dithiothreitol, 10 mM MgCl₂). 30 μg of each extract was loaded onto 10% polyacrylamide SDS gel (SDS-PAGE) and transferred to Immobilon-P membrane (Amersham). Protein expression was detected using enhanced chemiluminescence (ECL) assay. For this, β-actin which was standard protein commonly present in all cells, Caspsase-3 associated with apoptotic cell death, and AKT and Bcl-2 associated with cell survival were labeled with primary antibody (Santa Cruz, 1:1,000 dilution). Then, the membrane was washed with PBS and treated with a blotting solution to prevent a side reaction. Then, the membrane was incubated in a blocking solution containing Horseradish Peroxidase-conjugate anti-goat IgG (HRP) as a secondary antibody and then incubated with an ECL blotting reagent for 3 minutes. Chemiluminescence was detected using an X-ray film from 30 seconds to 20 minutes, and the results are shown in FIG. 11. Referring to FIG. 11, β-actin was expressed in all groups, whereas AKT and Bcl-2 associated with cell survival were expressed only in the normal group and the LDH-dose group. Caspase-3 associated with apoptotic cell death was strongly expressed in the RA-dose group and the LDH-RA dose group. This can be explained by RA-induced RXR/RAR dimerization. That is, a RXR/RAR dimer, formed by RA, is attached to an AP-1 binding site of genomic DNA during AP-1-mediated transcription and facilitates the transcription of interferon (IFN) localized in the downstream of the genomic DNA, thereby inducing apoptosis. Thus, even when LDH-RA is administered in a small dose, the entry and release of RA into cells through LDH can be facilitated, thereby enabling an effective pharmacological action of RA on the cells. This demonstrates the possibility of using LDH-RA as a promising anticancer drug.

### EXAMPLE 9

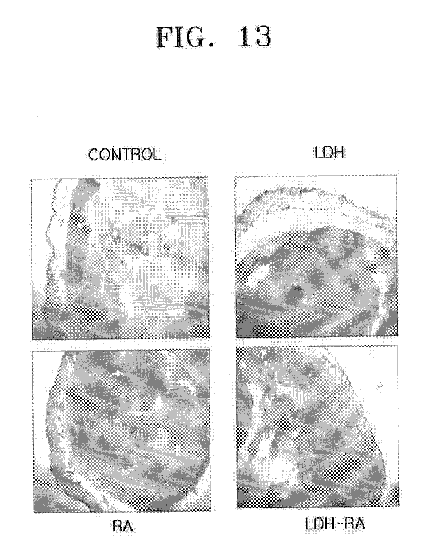

The CHX tumor cells were collected at 1×10⁷ cells/well and administered subcutaneously to the hind legs of athymic nude mice. Appearance of tumor mass was observed every week. Tumor masses appeared 3 weeks after the subcutaneous administration, and, when a tumor size was increased to 5 mm, one group of the mice was untreated (control group), and the other groups of the mice were treated as follows: a LDH-dose group with LDH (1 mg/ml), a RA-dose group with RA (0.5 mg/ml), and a LDH-RA dose group with LDH-RA (50 μg/ml). The LDH-dose group, the RA-dose group, and the LDH-RA dose group were further treated with LDH, RA, and LDH-RA, respectively, every two weeks for 8 weeks. The macro photographic images of tumor growth are shown in FIG. 12. Referring to FIG. 12, in the control group, a tumor size was increased to 30 mm after 8 weeks. In the LDH-dose group, a size reduction in tumor mass was slightly observed but tumor growth was not adversely affected. In the RA-dose group, a tumor size was reduced by about 20%. In the LDH-RA dose group, a tumor size was reduced by 80% or more. After then, the mice were anesthetized. Tumor tissues were cut, fixed in formalin, and cut into sections (5 μm thick) on a microtome. The sections were stained with hematoxylin/eosin (H/E) and examined with a microscope (50× magnification), and the results are shown in FIG. 13. Referring to FIG. 13, in the control group, tumor masses were found in almost all tissues, thereby causing growth retardation of tumor, resulting in necrosis. In the LDH-dose group, necrotic tumor tissues were observed, like in the control group. On the other hand, in the RA-dose group, necrosis was retarded due to slight inhibition of proliferation of tumor tissues, thereby resulting in a 15% reduction in tumor tissues. In the LDH-RA dose group, a tumor size was greatly reduced due to apoptosis of tumor tissues, and 85% or more tissue necrosis was observed, showing the prevention of tumor proliferation or growth. From the above results, it can be seen that LDH mediates the introduction of a LDH-RA hybrid into cells and the transport of the LDH-RA hybrid to small organelles, such as Golgi or lysosome, and when RA is released from LDH in an acidic pH of the small organelles, IFN synthesis is induced during transcription, thereby inducing the apoptotic cell death of tumor cells.

A layered metal hydroxide-retinoic acid (LMH-RA) hybrid according to the present invention stabilizes RA and guarantees the sustained-release property of RA (see the following Examples 1-2). The LMH-RA hybrid of the present invention also exhibits a higher anticancer efficacy than RA (see the following Examples 5-6). This is possible because LMH effectively facilitates RA delivery to a tumor cell. Furthermore, since RA toxicity problem, which may be caused when RA is used in a high dose, can be alleviated, the LMH-RA hybrid of the present invention has fewer RA-mediated side effects. Therefore, the LMH-RA hybrid of the present invention is very useful for a pharmaceutical composition for the treatment of cancers.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A pharmaceutical composition for the treatment of a cancer, comprising a hybrid of layered metal hydroxide and retinoic acid.

2. The pharmaceutical composition of claim 1, wherein the layered metal hydroxide is layered double hydroxide or hydroxy double salt.

3. The pharmaceutical composition of claim 1, wherein the retinoic acid is intercalated into an interlayer of the layered metal hydroxide using an ion exchange method, a coprecipitation method, or an adsorption method.

4. The pharmaceutical composition of claim 1, wherein the hybrid is represented by Formula 1 below:
[M²⁺₁₋ₓN³⁺ₓ(OH)₂][RAⁿ⁻]_{x/n}.yH₂O, (1)
wherein M²⁺ is a divalent metal cation selected from the group consisting of Mg²⁺, Ni²⁺, Cu²⁺, and Zn²⁺, N³⁺ is a trivalent metal cation selected from the group consisting of Al³⁺, Fe³⁺, V³⁺, Ti³⁺, and Ga³⁺, x is a value ranging from 0.1 to 0.5, RA is a retinoic acid or its derivative, n is a charge number of RA, and y is a positive number.

5. The pharmaceutical composition of claim 1, wherein the hybrid is represented by Formula 2 below:
[M²⁺(OH)₈][RAⁿ⁻]_{2/n}.yH₂O, (2)
wherein M²⁺ is a divalent metal cation selected from the group consisting of Mg²⁺, Ni²⁺, Cu²⁺, and Zn²⁺, RA is a retinoic acid or its derivative, n is a charge number of RA, and y is a positive number.

6. The pharmaceutical composition of claim 1, wherein the cancer is a liver cancer.
